(19) United States
(12) Patent Application Publication
(10) Pub. No.: US 2024/0294451 A1
(43) Pub. Date: Sep. 5, 2024

(54) PROCESS FOR PURIFICATION OF A MIXTURE INCLUDING DIOLS AND ACETALS

(71) Applicant: NOVAMONT S.P.A., Novara (IT)

(72) Inventors: Marco COTTI COMETTINI, Brusnengo (BI) (IT); Lorenzo QUATRALE, Novara (IT)

(21) Appl. No.: 18/258,626

(22) PCT Filed: Dec. 21, 2021
(86) PCT No.: PCT/EP2021/087113

§ 371 Date: Jun. 21, 2023

(30) Foreign Application Priority Data
Dec. 22, 2020 (IT) 102020000031979

(51) Int. Cl. C07C 29/80; C07C 29/143; C07C 29/88

(52) U.S. Cl. CPC C07C 29/80; C07C 29/143; C07C 29/88

(57) ABSTRACT

Process for purifying a mixture of a diol and an acetal, comprising hydrolysing the mixture with formation of the relative aldehydes/ketones and diols; and reducing the aldehydes/ketones present in the hydrolysis product by adding a reducing agent, resulting in a diol of the same species as present in the starting mixture.

The project leading to the invention was funded by the Bio Based Industries Joint Undertaking Public-Private Partnership under the European Union's Horizon 2020 research and innovation programme, under Grant Agreement No. 745012.

The present invention relates to a process for purifying diols, produced by fermentation and/or by chemical means, in order to increase their recovery, yield and quality.

Examples of diols are 1,2-ethanediol, 1,2-propanediol, 1,3-propanediol, 1,3-butanediol, 1,4-butanediol, 2,3-butanediol, 1,2-pentanediol, 1,3-pentanediol, 1,4-pentanediol, 1,5-pentanediol, 1,6-hexanediol, 1,7-heptanediol, 1,8-octanediol, 1,9-nonanediol, 1,10-decanediol, 1,11-undecanediol, 1,12-dodecanediol 1, 13-tridecanediol, 1,4-cyclohexanedimethanol, neopentylglycol, 2-methyl-1,3-propanediol, 3-methyl-1,5-pentanediol, 2-methyl-1,8-octanediol, 2,2-diethyl-1,3-propanediol, dianhydrosorbitol, dianhydromannitol, dianhydroiditol, cyclohexanediol, cyclohexanmethanediol, dialkylene glycols and polyalkylene glycols.

1,3-Butanediol (generally known as BG, 1,3-BG, 1,3-BDO, or 1,3-butylene glycol) is a four-carbon-atom diol that has two stereoisomers: R-1,3-BDO and S-1,3-BDO. The racemic mixture is commonly used in many industrial processes, for example as an organic solvent for food flavouring agents or as a reagent for the production of polyurethane resins and polyesters. Due to its low toxicity and high tolerability, it is also increasingly used in the cosmetics industry in personal care products, e.g. in the formulation of hair and bath products, eye and face make-up, perfumes, personal cleansing, shaving and skin care products. Optically active 1,3-BDO is also a widely used component of antibiotics, pheromones, fragrances and insecticides.

1,4-butanediol (generally known as 1,4-BDO, 1,4-BD or 1,4-butylene glycol), for example, is widely used as a monomer for the production of various types of products such as, for example, polyesters of the diacid-diol type or as an intermediate for the synthesis of compounds such as gamma-butyrolactone and tetrahydrofuran. Because of their mechanical and processing properties, polyesters comprising repeating units derived from a dicarboxylic acid and a diol are nowadays widely used in all fields where thermoplastic polymer materials such as films, moulded and blown articles, as well as fibres, are used. Moreover, it is preferable that the polyesters thus obtained are biodegradable, in particular according to EN 13432.

The chemical production of C2-C4 short-chain diols from fossil resources has been developed and optimized for decades. 1,3-BDO is conventionally produced by a chemical process involving the hydration of acetylene with formation of acetaldehyde, which is subsequently converted to 3-hydroxy butyraldehyde and reduced to form 1,3-BDO.

1,4-BDO can be synthesised by various chemical processes from petrochemical feedstocks: acetylene, via ethynylation with formaldehyde: butadiene, via acetylation or halogenation: propylene, via epoxidation or oxyacetylation: n-butane, via formation of maleic anhydride and its subsequent hydrogenation by various routes.

1,3-propanediol is primarily produced by the hydration of acrolein. An alternative route involves the hydroformylation of ethylene oxide to obtain 3-hydroxypropionaldehyde, which is subsequently hydrogenated to give 1,3-propanediol.

Due to decreasing fossil resources, fluctuating oil prices and increasing environmental problems, the production of C2-C4 diols from renewable sources through biological processes has attracted considerable interest.

Indeed, 1,4-BDO can be produced through fermentation processes from renewable sources such as carbohydrates, such as sugars and lignocellulosic biomass, or from synthetic gases (CO, CO2 and/or H2), directly (WO 2015/158716) or via the formation of bio-succinic acid (WO 2011/063055) and its subsequent hydrogenation, or through the formation of polyhydroxyalkanoate (WO 2011/100601).

Patent application WO 2015/158716 describes a process for the production of 1,4-BDO comprising fermentation in a culture medium by a microorganism having at least one metabolic pathway for the synthesis of 1,4-BDO, in which said culture medium comprises a mixture of glucose and sucrose. Similarly, WO 2010/127319 describes a fermentative process for producing 1,3-BDO from renewable sources.

1,3-propanediol can also be produced by fermentation, for example from glucose using a genetically modified strain of E. coli, as described in US patent 2008/176302, or from glycerol using bacteria belonging to the genus Clostridium (WO 2020/030775).

However, impurities, including by-products resulting directly from the synthesis process and/or from degradation reactions, which may also occur during the subsequent purification process in processes for producing diols from both chemical and renewable sources, are generally formed.

Removal of these impurities is necessary to ensure the use of diols in, for example, the cosmetics industry and the synthesis of diacid-diol polyesters. The higher the level of purity, the more monomers are sought after in these sectors.

Impurities typically include compounds such as aldehydes, hemiacetals and acetals, which cause colouration and instability over time of the diols produced and therefore must be removed.

In processes for producing 1,4-BDO from chemical sources such as, for example, the catalytic hydrogenation of maleic acid derivatives, typically the 1,4-BDO obtained comprises 4-hydroxy butyraldehyde, its cyclic hemiacetal 2-hydroxytetrahydrofuran, and the cyclic acetal 2-(4-hydroxy butoxy) tetrahydrofuran (HB-THF).

In the processes for the production of 1,4-BDO obtained by fermentation, one of the impurities most commonly present is the acetal HB-THF.

The formation of acetals in diol production processes is therefore a common reaction. This reaction may occur during purification processes. Such purification processes typically involve operations at high temperature and under dehydrating conditions, such as occur during distillation.

In processes for purifying 1,4-BDO, for example, because HB-THF may be produced by the oxidation of 1,4-BDO and such oxidation may occur under the conditions under which distillation operations are conventionally performed, the distillation itself may result in an increase in the amount of HB-THF in the final product. During distillation operations in a process for purifying 1,4-BDO, HB-THF is typically removed along with a light fraction, which comprises impurities having a boiling point equal to or below that of pure 1,4-BDO (including alcohols, aldehydes, and acetals). However, this removal is particularly difficult since HB-THF has a boiling point very close to that of 1,4-BDO and also forms an azeotrope with it.

Due to the difficulty of performing an efficient separation between the two compounds, in order to obtain high purity 1,4-BDO and to remove large quantities of HB-THF, the above-mentioned distillation operations require many separation stages and result in the removal of some of the 1,4-BDO itself in the light fraction, with consequent loss of product and decrease in plant productivity.

It is therefore necessary to find a compromise between the desired level of product purity and productivity.

Patent application US 2016/0244387 describes a process for the purification of 1,4-BDO involving treatment with complex hydrides, which are added to the 1,4-BDO composition to be purified. However, this treatment, while being able to reduce aldehydes to alcohols, does not allow complete removal of HB-THF from the final product since, being an acetal, it does not react in the presence of the reducing agent. In fact, the process in this example is only able to remove up to 60% of HB-THF, which is removed through distillation operations.

There are alternative processes to distillation, such as, for example, the one reported in patent EP0891315B1, which describes a catalytic hydrogenation process that allows 1,4-BDO with a reduced HB-THE content to be obtained. Such a process is however very complicated and expensive.

In order to overcome the problems described above, it has now been surprisingly discovered that it is possible to purify a mixture comprising at least one diol and at least one of its acetals through a relatively simple process, without having significant losses of product, at the same time obtaining high purity products which have an APHA colour index below 25 and are stable even at high temperatures and in an acid environment.

Indeed, a process has been identified that is able to purify a mixture comprising at least one diol and at least one acetal thereof through at least one hydrolysis operation and subsequent treatment with a reducing agent. Such a process makes it possible to purify diols with high efficiency, obtaining products characterised by greater purity and stability and, at the same time, to recover the diols converted into acetals, minimising their losses.

In the process according to the invention the acetals undergo hydrolysis, with formation of the corresponding aldehydes and/or ketones and the corresponding alcohols. The aldehydes and/or ketones obtained after hydrolysis of the acetals are subsequently reduced to alcohols by treatment with a reducing agent. The alcohols obtained after hydrolysis of the acetals and/or reduction of the aldehydes and/or ketones predominantly consist of the diol of the same species as is present in the starting mixture.

Thus, a first object of the present invention is a process for purifying a mixture comprising at least one diol and at least one acetal thereof, said process comprising the steps of:
(a) hydrolysis of the mixture comprising at least one diol and at least one acetal thereof, resulting in the formation of the relative aldehydes and/or ketones and the relative diols:
(b) reduction of the aldehydes and/or ketones present in the hydrolysis product in step (a) by the addition of a reducing agent, resulting in the diol of the same species as is present in the starting mixture.

According to one aspect, predominantly aldehydes are formed in the hydrolysis in step (a).

According to an alternative aspect, predominantly ketones are formed in the hydrolysis in step (a).

The mixture according to the invention comprises at least one diol selected from: 1,2-ethanediol, 1,2-propanediol, 1,3-propanediol, 1,3-butanediol, 1,4-butanediol, 2,3-butanediol, 1,2-pentanediol, 1,3-pentanediol, 1,4-pentanediol, 1,5-pentanediol, 1,6-hexanediol, 1,7-heptanediol, 1,8-octanediol, 1,9-nonanediol, 1,10-decanediol, 1,11-undecanediol, 1,12-dodecanediol, 1,13-tridecanediol 1,4-cyclohexanedimethanol, neopentylglycol, 2-methyl-1,3-propanediol, 3-methyl-1,5-pentanediol, 2-methyl-1,8-octanediol, 2,2-diethyl-1,3-propanediol, dianhydrosorbitol, dianhydromannitol, dianhydroiditol, cyclohexanediol, cyclohexanmethanediol, dialkylene glycols and polyalkylene glycols and mixtures thereof. Preferably the mixture comprises a diol selected from 1,2-ethanediol, 1,2-propanediol, 1,3-propanediol, 1,3-butanediol, 1,4-butanediol, 2,3-butanediol, 1,2-pentanediol, 1,3-pentanediol, 1,4-pentanediol, 1,5-pentanediol, 1,6-hexanediol, 1,7-heptanediol, 1,8-octanediol, 1,4-cyclohexanedimethanol, neopentylglycol, 2-methyl-1,3-propanediol, 3-methyl-1,5-pentanediol, 2,2-diethyl-1,3-propanediol, dianhydrosorbitol, dianhydromannitol, dianhydroiditol, cyclohexanediol, cyclohexanmethanediol, dialkylene glycols and mixtures thereof.

More preferably, the mixture comprises a diol selected from 1,4-BDO, 1,3-BDO and a mixture thereof. Even more preferably, the mixture comprises 1,4-BDO.

The process according to the invention may be used to purify a mixture comprising 1,4-BDO and at least one acetal thereof, even when 1,4-BDO is present in a mixture with 1,3-BDO. Said mixture of 1,4-BDO and 1,3-BDO can for example be obtained by the process described in Italian patent application 102020000013243.

According to a preferred embodiment, the diol in the mixture according to the invention comprises 1,4-BDO and its acetal comprises predominantly HB-THF.

The process according to the invention is particularly advantageous because it makes it possible to obtain high purity diols that are particularly suitable for use in polymerisation processes, where monomers are more and more sought after the higher their purity, following simple operations aimed at removing water, salts and reducing agent.

Thus a second object of the present invention is a 1,4-BDO composition, preferably obtained by the process for purifying a mixture comprising at least one diol and at least one acetal thereof above described, the said 1,4-BDO composition having an HB-THE content of less than 400 ppm, preferably less than 300 ppm, even more preferably less than 200 ppm, characterised by an APHA colour index of less than 25 preferably less than 15, even more preferably less than 10, which is advantageously stable even at temperatures above 150° C., preferably above 180° C. and in an acidic environment.

The above composition may be obtained through the process according to the invention.

The process according to the invention will be described in more detail below.

Hydrolysis step (a) is carried out in the presence of water in an amount exceeding 20%, preferably of 30% by weight or greater, relative to the total weight of the aqueous solution.

According to the aspect of the invention wherein the mixture comprises 1,4-BDO, the amount of water is advantageously greater than 50% by weight, for example 50 to 99% by weight, preferably 55 to 95% by weight, more preferably 75 to 90% by weight, relative to the total weight of the aqueous solution. According to the aspect of the invention wherein the mixture mainly comprises 1,3-BDO, the amount of water is advantageously lower than 70% by weight, for example 20 to 50% by weight, preferably 25 to 40% by weight, relative to the total weight of the aqueous solution.

Large amounts of water promote the hydrolysis reaction, but all the water must then be subsequently removed, with associated removal costs.

Advantageously, the hydrolysis can be conducted at a pH of 7 or below, preferably of between 3 and 7, preferably between 4 and 5.

Such pH values may also be obtained by the addition of an acid, preferably a strong mineral acid, which may, for example, be selected from orthophosphoric, sulfuric and hydrochloric acids.

The hydrolysis is conducted by keeping the solution stirred, preferably at a temperature of between 25 and 170° C., more preferably between 50 and 100° C., even more preferably between 70 and 95° C., for a time of between 1 minute and 240 minutes, preferably between 5 minutes and 120 minutes, even more preferably between 15 minutes and 40 minutes.

Related aldehydes and/or ketones and related alcohols are formed from the acetal through hydrolysis step (a). In the process of purifying a mixture comprising 1,4-BDO and at least one acetal thereof, typically HB-THF, for example, 4-hydroxy butanal and 1,4-BDO are formed from the hydrolysis of HB-THF.

The hydrolysis product obtained at the end of step (a) undergoes a reducing treatment in step (b). In said step (b) the aldehydes and/or ketones present in the hydrolysis product are reduced to alcohols by the addition of a reducing agent, resulting in the diol of the same species as is present in the starting mixture. The reducing agent is preferably a complex hydride.

Complex hydrides are those specified, for example, in Advanced Organic Chemistry, J. March, 4th edition J. Wiley & Sons, 1992, pages 910-917. Examples of complex hydrides are BH3, AlH3, LiBH4, NaBH4, KBH4, LiAlH4, NaAlH4, KAlH4, their forms in which some of the hydride hydrogens are replaced by anions such as in the form of alkylate, alkoxylate, acylate and the like, and Si hydrides such as Et3SiH.

The hydrides may include additives such as alkali metal hydroxides or alkaline earth metal hydroxides such as salts of Li, La and Ce in the form of halides, sulfates, phosphates or carboxylates.

Sodium borohydride and potassium borohydride are preferred.

Particularly preferred is sodium borohydride (of formula NaBH4 and also commonly known as sodium borohydride or sodium tetrahydroborate), e.g. in the form of aqueous alkaline solutions, sold under the name “Borol™ solution” by Rohm and Haas or under the name “VenPure™ solution” by Dow Chemical Company. These are in the form of an aqueous solution of 25-40% by weight NaOH and 10-12.5% by weight NaBH4.

The complex hydrides are advantageously used in approximately stoichiometric ratio or in slight excess of the carbonyl compounds present in the hydrolysis product from step (a). Such carbonyl compounds may, for example, be quantified by standard ASTM method E411.

The hydrolysis product obtained at the end of step (a), in reduction step (b) is preferably kept stirred at a temperature of between 25 and 170° C., more preferably between 50 and 120° C., even more preferably between 60 and 90° C., for a time of between 1 and 60 minutes, preferably between 2 minutes and 30 minutes, even more preferably between 3 and 15 minutes.

At the end of step (b), diols of the same species as is present in the starting mixture are obtained.

In step (b) of the process according to the invention, or in an optional subsequent step, the use of a basic pH may promote the hydrolysis of any esters present, further increasing diol recovery.

The process according to the invention may also be advantageously applied where the acetals content in the mixture comprising at least one diol and at least one acetal thereof, undergoing hydrolysis step (a), is particularly high. Indeed, it is particularly advantageous to separate a fraction enriched in at least one acetal from the mixture comprising at least one diol and at least one acetal thereof before hydrolysis step (a).

An enriched fraction is defined as a fraction having an acetals content of more than 1000 ppm, preferably more than or equal to 9000 ppm.

According to a preferred embodiment, the present invention therefore relates to a process for purifying a mixture comprising at least one diol and at least one acetal thereof, said process comprising a preliminary step of separating from said mixture a fraction enriched in at least one acetal, followed by the steps of:
(a) hydrolysis of said fraction enriched in at least one acetal, with formation of the relevant aldehydes and/or ketones and the relevant diols:
(b) reduction of the aldehydes and/or ketones present in the hydrolysis product from step (a) by the addition of a reducing agent, resulting in the diol of the same species as is present in the starting mixture.

The acetals separated in the enriched fraction undergo hydrolysis, with the formation of related aldehydes and/or ketones and related alcohols. The aldehydes and/or ketones obtained after hydrolysis of the acetals, or removed together with the fraction enriched in at least one acetal, are subsequently reduced to alcohols by treatment with a reducing agent. The alcohols obtained after hydrolysis of the acetals and/or reduction of the aldehydes and/or ketones or separated together with the fraction enriched in at least one acetal consist predominantly of diols of the same species as are present in the starting mixture.

This process makes it possible to purify the diols with high efficiency, obtaining products characterised by greater purity and stability and, at the same time, to recover both the diols converted into acetals and those separated together with the fraction enriched in at least one acetal, minimising losses.

The fraction enriched in at least one acetal is obtained through a preliminary separation step, preferably carried out by techniques known to those skilled in the art, such as distillation, fractional crystallisation, evaporation.

According to a preferred aspect, separation of the fraction enriched in at least one acetal takes place by distillation. Said fraction enriched in at least one acetal separated by distillation may be the heavy or light fraction with respect to the diol or diols predominantly present in the mixture.

The “light fraction” refers to the fraction removed from the distillation column heads and typically comprises a mixture of compounds with a boiling point equal to or below that of the pure diol (so-called “light compounds”). In the case where the diol comprises 1,4-BDO, examples of light compounds are: 4-hydroxybutanal, 4-hydroxybutyl acetate, 2-(4-hydroxy butoxy)-tetrahydrofuran (HB-THF), gamma-butyrolactone, 4-hydroxybutyl acetate and 1,4-butanediol diacetate.

The “heavy fraction” refers to the fraction removed from the bottom of the distillation columns and typically comprises a mixture of compounds with a higher boiling point than the pure diol (so-called “heavy compounds”). In the case where the diol comprises 1,4-BDO, examples of heavy compounds are organic compounds that may have undergone a degradation process, 2-pyrrolidone and 1,6-hexanediol.

In the case where the diol comprises 1,4-BDO, the fraction enriched in at least one acetal is advantageously the light fraction, which is separated from the mixture comprising 1,4-BDO and at least one acetal thereof by a distillation operation, preferably conducted at a temperature between 50 and 250° C., more preferably between 100 and 170° C., and a head pressure between 5 and 200 mbar, more preferably between 20 and 80 mbar.

In this application the operating pressure of the distillation columns is understood to be measured in absolute millibars (mbar). 1 mbar corresponds to 100 Pascal.

The mixture comprising at least one diol and at least one acetal thereof may be derived from a process for purifying diols, preferably from previous distillation operations typically carried out to remove solvent and heavy compounds. The process according to the invention is therefore advantageously applied to processes for purifying diols comprising distillation operations. Prior to the step of separating the enriched fraction from the mixture comprising at least one diol and at least one acetal thereof, the purification process according to the present invention may advantageously comprise one or more preliminary distillation operations to remove the solvent, preferably water, from the mixture comprising the diol and a fraction comprising heavy compounds (so-called heavy fraction).

At the end of step (b) the process according to the invention makes it possible to obtain diols of the same species as are present in the starting mixture, present therein or formed after the hydrolysis of acetals and/or the reduction of aldehydes and/or ketones, which would otherwise be removed together with the acetals, with consequent loss of product.

Where the initial mixture contains diols from a synthesis process starting from a renewable source, advantageously the diols obtained from the process according to the invention can be subjected to further optional solid/liquid separation and/or concentration and/or distillation operations, aimed at removing the water, salts and reducing agent introduced in steps (a) and (b) of the process according to the invention, obtaining high purity diols, suitable for use in polymerisation processes, and increasing the yield of the production plants.

The separation operations may be for example carried out by one or more of decantation, centrifuging, filtration, microfiltration, nanofiltration, ultrafiltration, ion exchange, osmosis, other suitable solid/liquid separation techniques and combinations thereof.

The concentration operations may for example be chosen from evaporation and/or reverse osmosis.

The aqueous solution obtained at the end of step (b) may for example undergo one or more treatments with ion exchange resins, as described in patent application WO 2019/102030.

Said resins may be cationic and anionic exchange resins.

Cation exchange resins are generally selected from the group consisting of resins derived from strong acids (e.g. sulfonate groups) or weak acids (e.g. carboxylate groups) and preferably contain functional groups selected from sulfonate groups. Non-limiting examples of cation exchange resins include, for example, the resin commercially available under the brand name DOWEX® 88 or DOWEX® 88 MB.

Anion exchange resins are generally selected from the group consisting of resins derived from strong bases (e.g. quaternary amine groups) or weak bases (e.g. tertiary amine groups) and preferably contain functional groups selected from the quaternary amine groups. Non-limiting examples of anion exchange resins include, for example, the resin commercially available under the brand name DOWEX® 22.

The order of succession of the steps in the cationic and anionic exchange resins is not particularly limiting. One or more passes through the cation exchange resins may precede or succeed one or more passes through the anion exchange resins. Preferably, the one or more passes through the cation exchange resins precede one or more passes through the anion exchange resins.

The aqueous solution obtained at the end of step (b) or after the passage through the ion exchange resins or between different passages through said resins, may undergo concentration operations using techniques known to those skilled in the art.

The aqueous solution obtained after passing through the ion exchange resins and after concentration may undergo distillation.

The distillation operations may be conducted by appropriately dimensioning the distillation system to effectively purify diols having different contents of impurities.

The number of distillation operations and the number of columns for each operation is not particularly limiting.

Each of the distillation operations may be conducted independently according to techniques known in the state of the art employing different types and configurations of distillation columns. For example, the distillation columns may comprise random-fill, structured-fill, flat-fill, random-fill and structured, random-fill and flat-fill, or structured-fill and flat-fill sections.

Filled, advantageously structured-fill columns are preferred.

Each of the distillation operations may be conducted through a single column or train of columns, or through more integrated configurations that allow more than two streams to be obtained from each column, for example with side extractions of product or with the insertion of vertical baffles to minimise the number of columns and ancillary equipment.

The distillation operations according to the present invention are preferably carried out by reducing or minimising exposure of the compounds to high temperatures. In fact both the products and the impurities therein may undergo thermal or chemical degradation due to heating during distillation. Operation of the distillation columns at reduced pressure (below atmospheric pressure) or vacuum is preferred as it lowers the boiling temperature of the mixture in the distillation column and allows the distillation column to be operated at lower temperatures.

Persons skilled in the art will be able to adjust the operating conditions at each stage of the process to the type of column(s) used. A common vacuum system may be used with some or all of the distillation columns to achieve reduced pressure, or each column may have its own vacuum system.

The pressure in a distillation column may be measured at the top or in the condenser, at the bottom or at the base or anywhere in between. The different distillation columns in the process according to the invention may operate at different pressures.

According to one embodiment, the aqueous solution obtained at the end of step (b) is subjected to evaporation and ion exchange treatment.

Where the initial mixture contains 1,4-BDO, at the end of these operations it is possible to obtain 1,4-BDO with an HB-THF content of less than 400 ppm, preferably less than 300 ppm, more preferably less than 200 ppm, even more preferably less than 180 ppm.

The 1,4-BDO thus obtained is also advantageously characterised by an APHA colour index of less than 25, preferably less than 10, which is even more advantageously stable even at temperatures above 150° C., preferably above 180° C. and in an acid environment.

These characteristics make the 1,4-BDO suitable for use, for example, in the synthesis of polyesters of the diacid-diol type, in which a BDO purity in excess of 98%, preferably even in excess of 99%, and a stability such that degradation is prevented during the process of synthesising such polyesters, are required.

Said 1,4-BDO may be produced by a fermentation process, conducted for example according to the process described in WO 2015/158716, in which 1,4-BDO is synthesised from at least one sugar, preferably glucose and optionally one or more sugars other than glucose, in the presence of one or more microorganisms having at least one metabolic pathway for synthesising 1,4-BDO.

The conversion of sugars to 1,4-BDO in a fermentation process is typically below 100% because, in addition to the diol, intermediates (by-products) of the metabolic pathways used by the microorganisms to produce 1,4-BDO are also produced, and these can accumulate in the fermentation broth.

Furthermore, at the end of fermentation, the organisms or cells comprising the cellular biomass present in the fermentation broth may be subjected to deactivation or killing, for example by thermal means, causing residues and cellular metabolites to be released into the fermentation broth.

The 1,4-BDO obtained from the above fermentation process and present in the fermentation broth therefore typically undergoes solid/liquid separation operations to remove one or more elements including microorganisms, cellular residues, any unreacted sugars, by-products, mineral salts, metabolites and components of the culture medium not assimilated or metabolised by said microorganisms from the fermentation broth, resulting in a mixture containing 1,4-BDO.

Said separation operations may be carried out by one or more operations chosen from decantation, centrifuging, filtration, microfiltration, nanofiltration, ultrafiltration, ion exchange, osmosis, other suitable solid/liquid separation techniques and combinations thereof, as described in patent applications WO 2019/102030 and Italian patent application 102020000013243.

In addition to separation, the fermentation broth or mixture comprising 1,4-BDO may undergo concentration operations designed to change the content of solvent (e.g. water). Such operations are for example chosen from evaporation and reverse osmosis.

Following the separation and concentration operations mentioned above, the mixture comprising 1,4-BDO may be subjected to one or more distillation operations before undergoing the process according to the present invention.

The following examples illustrate the present invention for non-limiting purposes.

### METHODS OF MEASUREMENT

### APHA Colour Index

The APHA colour index was determined by spectrophotometry, according to the ASTM D1209-05 standard, before and after thermal stability tests.

### Thermal Stability Test

The thermal stability test was conducted by keeping 50 g of sample in an open glass flask stirred at 375 rpm and heating it until the internal temperature of 200° C. was reached, with the possibility of air exchange with the atmosphere. Once this temperature had been reached, the sample was held at 200° C., without stirring, for two hours. Immediately after cooling, thermal stability was evaluated by spectrophotometric measurement of its APHA colour, according to standard ASTM D1209-05.

### Stability Test in Acid Environment

The stability test in acid environment was performed in a 2 ml vial by adding 0.5 ml of 37% hydrochloric acid to 0.5 ml of sample and shaking vigorously. The sample was kept at room temperature for 24 hours, following which colour development was checked. The test result was considered positive in absence of colouration (e.g. Reflectance in the UV and/or visible region of 90% or above 90%), negative in case of colour change from yellow to brown (e.g. R<90% at about 496 nm), indicating instability.

### Quantification of Impurities

Analysis of the impurities content was performed using a gas chromatograph equipped with a column suitable for the analysis of volatile products (e.g. Zebron ZB-624 30 m×0.32 mm×1.40 μm column) and a single quadrupole mass spectrometer, using the following operating conditions:
Oven temperature programme: 50° C. (5 min)—240° C. (10° C./min)—240° C. (5 min)
Injection volume: 1 μL (hot needle injection—preheating time 2″)
Split injection with split ratio 1:10
Injector temperature: 270° C.
Carrier gas: He, 1.2 ml/min
Transfer line: 250° C.
Ion source temperature: 250° C.
Ionisation mode: EI
Full Scan acquisition: 33 to 650 uma with delay time of 5 min and filament off between 15.75 min and 16.75 min corresponding to arrival of the BDO at the detector.

The species present were quantified using an internal standard and considering a response factor of 1 for each impurity.

### Quantification of Carbonyl Compounds

Analysis of the content of impurities having carbonyl groups was performed using standard method ASTM E411 (Standard Test Method for Trace Quantities of Carbonyl Compounds with 2,4-Dinitrophenylhydrazine), using ethanol instead of methanol in preparation of the reagent solutions and constructing the calibration line with 4-hydroxy butanal standard.

### EXAMPLES

### Example 1

### Step (a)

A mixture comprising 93.9% by weight of 1,4-BDO and 10230 ppm of HB-THF was diluted to 7% by weight in water previously heated to 80° C. The aqueous solution, of pH 4.5, was held at 80° C. with stirring for 1 hour.

### Step (b)

The content of compounds having carbonyl groups present in the aqueous solution obtained at the end of hydrolysis step (a) was quantified as indicated above by the ASTM method. 8130 ppm of Venpure™ Solution (NaBH4 12.03% by weight, NaOH 39.32% by weight and water 48.65% by weight) with respect to the organic fraction, i.e. in slight excess of the stoichiometric content of carbonyl groups, was added to the aqueous solution. The aqueous solution was then held at 70° C. with stirring for 30 minutes. The aqueous solution obtained at the end of step (b) was concentrated, heating it at 70° C. at a pressure of 20 mbar, in order to remove the water present.

Subsequently the concentrated aqueous solution underwent fractional distillation in a 500 ml glass flask at a pressure of 33 mbar, reaching an internal temperature of about 138° C. At the end of the fractional distillation, a head fraction, a middle fraction comprising 1,4-BDO and a tail fraction were obtained.

The HB-THF and 1,4-BDO content, the APHA colour index and the results of thermal and acid stability tests on the 1,4-BDO obtained in the central fractional distillation fraction (fraction equal to about 80% by weight of the concentrated aqueous solution distilled) are reported in Table 1.

### Comparative Example 2

The same mixture as in Example 1 was subjected to fractional distillation directly, without performing hydrolysis and reduction steps (a) and (b), using the conditions described in Example 1.

The HB-THF and 1,4-BDO content, the APHA colour index and the results of thermal and acid stability tests on the 1,4-BDO obtained in the central fractional distillation fraction (fraction equal to about 80% by weight of the concentrated aqueous solution distilled) are reported in Table 1.

### Comparative Example 3

The same mixture as in Example I was subjected to reduction step (b) under the conditions of Example 1 but without performing hydrolysis step (a). The subsequent fractional distillation was carried out using the conditions described in Example 1.

The HB-THF and 1,4-BDO content, the APHA colour index and the results of thermal and acid stability tests on the 1,4-BDO obtained in the central fractional distillation fraction (fraction equal to about 80% by weight of the concentrated aqueous solution distilled) are reported in Table 1.

TABLE 1

| Central distillation | | Comparative | Comparative |
|---|---|---|---|
| fraction | Example 1 | Example 2 | Example 3 |
| % by weight of 1,4-BDO in | 98.7 | 96.9 | 97.7 |
| total central fraction | | | |
| ppm HB-THF | 487 | 11963 | 7797 |
| APHA colour | 21 | 24 | 16 |
| APHA colour after thermal | 26 | >450 | 90 |
| stability test | | | |
| Acid stability test | positive | negative | negative |

The percentage by weight of 1,4-BDO was calculated by subtracting the percentage sum of impurities quantified by the method described above from 100.

The examples clearly show that, using the process according to the invention (Example 1), characterised by a hydrolysis step and a reduction step, it is possible to remove HB-THF acetal with high efficiency, more than in the reduction step alone (Example 3).

Furthermore, in comparative Example 2, in which the light distillation fraction underwent neither hydrolysis nor reduction, not only is there no removal of acetals, but the fractional distillation also resulted in the formation of additional HB-THF, probably from aldehydes that were not reduced.

The process according to the invention therefore allows a greater amount of 1,4-BDO, which is also characterised by higher purity (98.7% in Example 1, 97.7% and 96.9% in Comparative Examples 2 and 3 respectively) to be recovered.

The BDO obtained by the process according to the invention is moreover advantageously stable even after being subjected to high temperatures and in an acid environment, as shown by the values obtained for the APHA colour index evaluated under the different conditions. On the other hand, the BDO obtained in Comparative Examples 2 and 3, although characterised by an APHA colour index of less than 25, is not stable when subjected to high temperatures and an acid environment.

1. A process for purifying a mixture comprising at least one diol and at least one acetal thereof, said process comprising the steps of:
(a) hydrolysis of the mixture comprising at least one diol and at least one acetal thereof, resulting in the formation of the relative aldehydes and/or ketones and the relative diols; and
(b) reduction of the aldehydes and/or ketones present in the hydrolysis product from step (a) by the addition of a reducing agent, resulting in a diol of the same species as is present in the starting mixture.

2. The process according to claim 1 in which the mixture comprises a diol selected from: 1,2-ethanediol, 1,2-propanediol, 1,3-propanediol, 1,3-butanediol, 1,4-butanediol, 2,3-butanediol, 1,2-pentanediol, 1,3-pentanediol, 1,4-pentanediol, 1,5-pentanediol, 1,6-hexanediol, 1,7-heptanediol, 1,8-octanediol, 1,9-nonanediol, 1,10-decanediol, 1,11-undecanediol, 1,12-dodecanediol, 1,13-tridecanediol 1,4-cyclohexanedimethanol, neopentylglycol, 2-methyl-1,3-propanediol, 3-methyl-1,5-pentanediol, 2-methyl-1,8-octanediol, 2,2-diethyl-1,3-propanediol, dianhydrosorbitol, dianhydromannitol, dianhydroiditol, cyclohexanediol, cyclohexanmethanediol, dialkylene glycols and polyalkylene glycols and mixtures thereof.

3. The process according to claim 2 in which the mixture comprises 1,4-BDO.

4. The process according to claim 3 in which the acetal comprises predominantly HB-THF.

5. The process according to claim 1, comprising a preliminary step of separating a fraction enriched in at least one acetal from said mixture comprising at least one diol and at least one acetal thereof.

6. The process according to claim 5, in which said enriched fraction has an acetal content of more than 1000 ppm.

7. The process according to claim 5, in which the preliminary separation step is performed by distillation.

8. The process according to claim 1 in which hydrolysis step (a) is carried out in the presence of water in an amount exceeding 20% by weight with respect to the total weight of the aqueous solution obtained.

9. The process according to claim 1 in which the mixture comprises 1,4-BDO and the hydrolysis step (a) is carried out in the presence of water in an amount exceeding 50% by weight with respect to the total weight of the aqueous solution obtained.

10. The process according to claim 8 in which the hydrolysis is conducted at a pH of 7 or below, preferably from 3 to 7.

11. The process according to claim 1, in which the reducing agent in step (b) is a complex hydride.

12. The process according to claim 11, in which the reducing agent in step (b) is sodium borohydride.

13. A 1,4-BDO composition having an HB-THE content of less than 400 ppm, characterised by an APHA colour index of less than 25, which is stable even at temperatures above 150° C. and in acid environment.

14. The process according to claim 2, comprising a preliminary step of separating a fraction enriched in at least one acetal from said mixture comprising at least one diol and at least one acetal thereof.

15. The process according to claim 3, comprising a preliminary step of separating a fraction enriched in at least one acetal from said mixture comprising at least one diol and at least one acetal thereof.

16. The process according to claim 4, comprising a preliminary step of separating a fraction enriched in at least one acetal from said mixture comprising at least one diol and at least one acetal thereof.

17. The process according to claim 2 in which hydrolysis step (a) is carried out in the presence of water in an amount exceeding 20% by weight with respect to the total weight of the aqueous solution obtained.

18. The process according to claim 3 in which hydrolysis step (a) is carried out in the presence of water in an amount exceeding 20% by weight with respect to the total weight of the aqueous solution obtained.

19. The process according to claim 4 in which hydrolysis step (a) is carried out in the presence of water in an amount exceeding 20% by weight with respect to the total weight of the aqueous solution obtained.

20. The process according to claim 5 in which hydrolysis step (a) is carried out in the presence of water in an amount exceeding 20% by weight with respect to the total weight of the aqueous solution obtained.
